(19) United States
(12) Patent Application Publication
(10) Pub. No.: US 2010/0009336 A1
(43) Pub. Date: Jan. 14, 2010

(54) System and method for bacterial vaginosis testing

(76) Inventor: Shannon E. Sullivan, Benicia, CA (US)

Correspondence Address: C. Bart Sullivan, 1543 Sherman Dr., Benicia, CA, 94510, US

(21) Appl. No.: 12/459,748

(22) Filed: Jul. 7, 2009

Related U.S. Application Data
(60) Provisional application No. 61/134,590, filed on Jul. 11, 2008.

(51) Int. Cl. C12Q 3/00; C12M 1/34

(52) U.S. Cl. 435/3; 435/286.2

(57) ABSTRACT

A method and system for testing fluids from a patient is provided. In one embodiment, a portable device is configured with one or more testing modules adapted perform one or more tests on a fluid sample in order to determine if a patient has bacterial vaginosis. A timer module coupled to a sample transport is configured to move a vaginal fluid sample between one or more testing modules at various stages along a predetermined testing path. In an embodiment, the timer module is configured to move the vaginal fluid sample between the testing modules along a circular testing path. The vaginal fluid sample may be processed in sequential order over predetermined time periods with respect to the processing performed by the testing module in order to detect the presence of, or lack of, Lactobacilli in the patient's vaginal flora to determine whether or not the patient has bacterial vaginosis.

### CROSS-REFERENCE TO RELATED APPLICATIONS

This application claims priority to U.S. Provisional Patent Application No. 61/134,590, entitled “System and Method for Bacterial Vaginosis Testing”, filed Jul. 11, 2008, which is hereby incorporated by reference in its entirety for all purposes.

### BACKGROUND

The present invention relates to the field of medical testing, and in particular to apparatus and methods for portable testing patients for bacterial vaginosis.

Animals often carry beneficial bacteria in a symbiotic relationship. Such beneficial bacteria often provide a benefit to the human host. For example, Lactobacillus is often present in humans in the vagina and the gastrointestinal tract where they are symbiotic and generally make up a small portion of flora. In the symbiotic relationship, Lactobacillus produces lactic acid to provide an acidic environment. Such an acidic environment helps reduce harmful bacteria.

The lack of Lactobacilli in the vaginal flora often causes many types of health issues. For example, the lack of Lactobacilli can lead to health issues such as sepsis, susceptibility to greater infection, and general discomfort. Further, infection could cause absesses in the urogenital system, and has been linked to miscarriage. An imbalance of naturally occurring Lactobacilli in the vaginal flora often leads to Bacterial Vaginosis (BV). BV is the most common cause of vaginal infection, and is the most common vaginal infection in women of childbearing age. A change in normal bacterial flora, including the reduction of lactobacillus often caused by the use of antibiotics or pH imbalance, allows harmful bacteria to gain a foothold and multiply. In turn these harmful bacteria produce toxins which affect the body's natural defenses and make re-colonization of healthy bacteria more difficult.

BV is currently diagnosed either by using a speculum examination and/or a clinical examination. For the speculum examination, the healthcare uses several separate tests to detect BV. For example, swabs of vaginal fluid discharge to test for a characteristic smell, often called the “whiff test” by adding a small amount of an alkali to a microscope slide that has been swabbed with the discharge. If the health care provided detects a ‘fishy’ odor, then the patient may have BV. Additionally, the healthcare provider often uses litmus paper to check for the loss of acidity which is a symptom of BV. A positive result for BV would be a pH of over 4.5. Finally, the healthcare provider also epithelial cells coated with bacterial called ‘Clue cells’ which provide clues to the discharge. The epithelial cells can be seen under microscopic examination of the discharge. For a clinical test, BV may be tested using the Amsel criteria or using a Gram stained vaginal smear, with different types of criteria such as the Hay/Ison criteria or the Nugent criteria, to determine if the patient has BV. Unfortunately, such healthcare provider and clinical testing requires tests be done in a medical office, hospital setting, or a laboratory, therefore requiring the patient to travel to the medical office, hospital or laboratory for diagnosis, or may require the patient visit a traveling clinic. In many parts of the world where it is difficult or often impossible for patients to obtain medical treatment, and where traveling clinics and healthcare providers are far and few between, such BV testing is generally unavailable.

Although BV is often considered a nuisance infection, untreated BV may cause serious complications, such as increased susceptibility to sexually transmitted infections including HIV, an increase a woman's susceptibility to other STDs, such as Chlamydia and gonorrhea, and may present other complications for woman that are pregnant. Further, BV has been associated with an increase in the development of infections such as Pelvic inflammatory disease (PID) following surgical procedures such as a hysterectomy or abortion.

Therefore, there is a need for a portable and simpler system and method for bacterial vaginosis testing that overcomes the issues discussed above.

### BRIEF SUMMARY

Embodiments of the invention provide a method and portable system for bacterial vaginosis testing. In one embodiment, a user enters a sample into the portable system for bacterial vaginosis testing having a plurality of testing modules configured to test the pH and gram test the sample fluid. The fluid sample is configured to display a color, such as purple, if the pH is between two pH thresholds defining a lactic acid range, and provide a different indicator otherwise. After further processing, the fluid sample is configured to provide a color, such as violet, or another indicator to indicate a threshold amount of Lactobacillus is present, and provide a different indicator if Lactobacillus is less than the threshold amount.

In one embodiment, the present invention provides a portable system for bacterial vaginosis testing which includes a portable enclosure and a plurality of testing modules disposed within the enclosure and disposed along a testing path. Each testing module may be configured to perform at least one portion of a bacterial vaginosis test on a fluid sample. A sample transport member is disposed within the enclosure and configured to support the fluid sample and position the fluid sample within each of the plurality of testing modules. A timer module may be coupled to the sample transport member configured to move the sample transport over a predetermined time period about the testing path in response to the timer module.

In one embodiment, the present invention provides a method of a user-self test for processing fluid samples from a patient to determine if the patient has bacterial vaginosis. The method includes providing a fluid sample to a portable testing device, wherein the portable testing device comprises at least two testing modules disposed along a testing path and a test transport member configured to support the fluid sample, moving the transport member and the fluid sample between the at least two testing modules along the testing path, processing the fluid sample with the at least two testing modules, determining from the bacteria within the processed fluid sample whether the patient has bacterial vaginosis, and providing a display to a user thereof indicating the results of the fluid sample processing.

In one embodiment, the present invention provides a computer program product having a computer readable medium storing a set of code modules which when executed by a processor of a computer system cause the processor to process fluid samples from a patient to determine if the patient has bacterial vaginosis. The computer readable medium includes code for providing a fluid sample to a portable testing device, wherein the portable testing device comprises at least two testing modules disposed along a testing path and a test transport member configured to support the fluid sample, code for moving the transport member and the fluid sample between the at least two testing modules along the testing path, code for processing the fluid sample with the at least two testing modules, code for determining from the bacteria within the processed fluid sample whether the patient has bacterial vaginosis, and code for providing a display to a user thereof indicating the results of the fluid sample processing.

These and other embodiments of the invention are described in further detail below.

### BRIEF DESCRIPTION OF THE DRAWINGS

FIG. 1 is a simplified illustration of a portable bacterial vaginosis testing system in accordance with embodiments of the invention;

FIG. 2 is a cut-away illustration of the portable bacterial vaginosis system in accordance with embodiments of the invention;

FIG. 3 is a cut-away illustration of the portable bacterial vaginosis system in accordance with embodiments of the invention;

FIG. 4 is a high-level diagram illustrating an embodiment of testing driver for bacterial vaginosis testing in accordance with embodiments of the invention;

FIG. 5 is a high-level block diagram illustrating a memory used for storing instructions used with testing driver for bacterial vaginosis testing in accordance with embodiments of the invention; and

FIG. 6 is a high-level diagram illustrating an embodiment of a method of testing for bacterial vaginosis in accordance with embodiments of the invention.

### DETAILED DESCRIPTION OF EXEMPLARY EMBODIMENTS

Embodiments of the invention are directed to a system and method for bacterial vaginosis testing. In one embodiment, a portable bacterial vaginosis testing system is disclosed. The portable bacterial vaginosis testing system may include one or more testing modules configured to perform one or more tests to determine if a patient has bacterial vaginosis. A sample of a patient's vaginal fluid may be supported by a testing swab that in one embodiment, exposes the fluid sample to the one or more testing modules over varying periods of time in a predetermined order. The results of the one or more testing modules fluid sample processing may be used to provide an indication that the patient has, or does not have, bacterial vaginosis. Advantageously, such a portable test would help prevent misdiagnosis and encourage patients to seek treatment early and often.

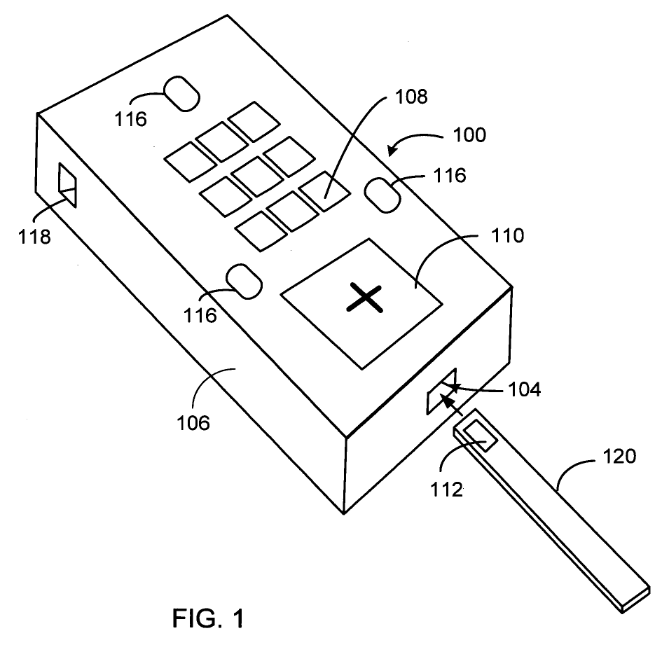

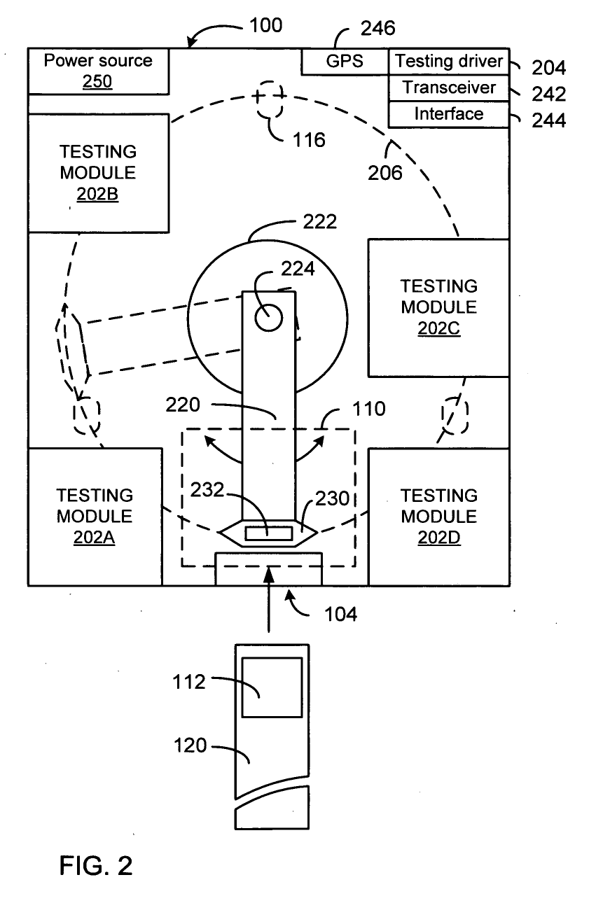

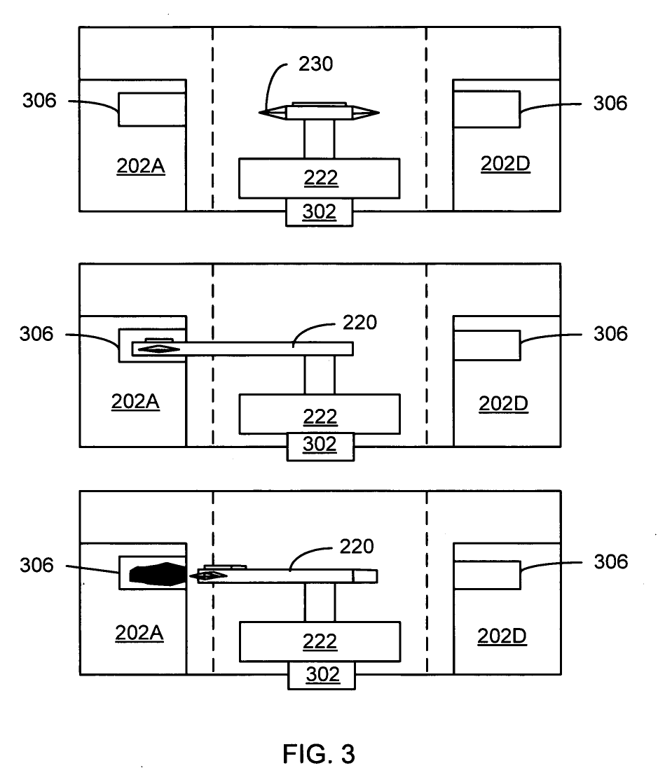

FIG. 1 illustrates a portable bacterial vaginosis (BV) testing system 100 according to an embodiment of the present invention. FIGS. 2 and 3 are cut-way views of the portable BV testing system 100. BV testing system 100 depicted in FIGS. 1 and 2 is merely illustrative of an embodiment incorporating the present invention and is not intended to limit the scope of the invention as recited in the claims. One of ordinary skill in the art would recognize other variations, modifications, and alternatives.

In one embodiment, BV testing system 100 may include an enclosure body 106 configured to be a hand-held portable device. As described further below, BV testing system 100 may include a display 110, one or more view ports 116, input device 108 such as a keyboard for data input, and I/O port 118, for data input and output. BV testing system 100 may be configured to receive one or more fluid samples via a fluid sample port. Fluid samples may be gathered using a fluid sample member 120 on a sample-gathering region 112 and placed onto a sample-holding region 232 of a sample transport 220. Fluid sample member 120 may be virtually any type of sampling device capable of gathering fluid sample. For example, fluid sample member 120 may be a swab having a light gel layer such as an oligo-gel matrix section disposed thereon to ensure attachment of bacteria. Alternatively, a quick drying adhesive could also be incorporated in lieu of the light gel layer.

As described further below, display 110 may either an electronic display, or may be a viewing window. For example, display 110 may be configured to allow a user to see a result via a processor output. In other embodiments, display 110 may be configured to allow a user to view a processed sample to see a color, indicator, etc., similar to, for example, a pregnancy test indicator. View ports 116 may be configured to allow a user to view a sample at various stages along a sample-testing path. Such viewing allows a user to see for example the results of a pH test, gram test, and the like.

In some embodiments, BV testing system 100 includes one or more testing modules 202A-D, used to test vaginal fluid samples. Sample transport 220 may be coupled to a timer module 222. Sample transport 220 may be configured to rotate in response to timer module 222 such that a fluid sample is moved between modules 202A-D at various times in order to process the sample in preprogrammed stages. For example, sample transport 220 may be an elongated sample member extending from a shaft of timer module 222. As timer module 222 operates, sample transport 220 movies the fluid sample between testing modules placed within a sample path 206 to carry out a sequence of tests and processes on the fluid sample. In one embodiment, the sample path 206 is circular, however, the sample path 206 may be non-circular. For example, the sample path may be straight, oval, rectangular, or irregular, where the sample transport is coupled to an adjustable sample transport 220 that is configured to follow the sample path 206. In another example, the sample path 206 may be defined by a track such as a rail, slot, and the like, that the sample transport 220 is configured to follow.

In one embodiment, timer module 222 is a mechanical timer (e.g., an egg timer), mechanical clock, or the like, configured to rotate sample transport 220 in a linear circular motion similar to a hand of a clock about a shaft 224. The timer module 222 may be a spring driven device that is pre-wound, or wound by operating a winding portion 302.

As the mechanical timer 222 may operate linearly, testing modules 202A-D may be placed along the sample path 206 such that the sample is processed at the correct time for a particular sample process. For example, given a complete bacterial vaginosis test takes twenty minutes to complete all of the tests, and the time lag between a first test and a second test is five minutes, testing module may be positioned along the sample path 206 such that as the sample transport 220 rotates, a first test may be performed at testing module 202A at a first time (e.g., zero minutes), and a second test may be performed at testing module 202B at five minutes after the first test. Therefore, in this embodiment, testing times may be relative to the positions of the testing modules 202A-D along the sample path 206. Such positioning of the testing modules 202A-D allows the sample to be processed in accordance to a preplanned testing sequence. Advantageously, using a linear timer and placing testing modules at various positional offsets along the sample path, allows timer module 222 to be part of, or responsive to, a mechanical timer that does not rely on external electrical power.

In another embodiment, timer module 222 may be an electronically controllable timer configured to move the sample transport 220 to the appropriate testing modules at preplanned times. For example, timer module 222 may be an electrical timer controlled by, a processor or the like, described herein, to move the sample transport 220 and therefore the sample between testing modules 202A-D at preprogrammed times.

Testing modules 202A-D may be configured to receive and process one or more fluid samples either passively, or may process samples actively under electronic control. For example, testing modules 202A-D may contain fluids, reagents, chemicals, pH strips, and the like to process a sample through contact and immersion, and/or may use electronic sensors to test such fluid. In one embodiment, to test bacterial vaginosis, testing modules 202A-D may be grouped according to a specific testing regime. For example, testing modules 202A-D may be grouped in different stages according to the test being performed such as a bacterial vaginosis test. In one embodiment, testing modules 202A-D may hold more than one testing compartment and are adapted to perform at least one test of a fluid sample according to bacterial vaginosis testing to detect the presence of healthy or pathogenic bacteria in the vaginal cavity. For example, testing modules 202A-D may have one or more compartments used to dry the sample, check for pH, provide a CV I ionic solution also known as “Grams iodine”, hold a number of solutions such as ethanol, water, etc.

In some embodiments, sample transport 220 may be configured with a puncture end 230 to puncture membranes or walls 306 used to seal portions of the testing modules 202A-D to premature exposure and potential contamination from external elements such as air, before a fluid test is initiated. Sample transport 220 may be configured with a puncture member 230 used to break such seals and/or walls. Puncture member 230 may be configured as part of sample transport 220, or may be a separate part attached thereto. Such seals and walls 306 may be made of materials such as rubber, plastic, paper, or the like, configured to seal, and that may be punctured.

So that BV testing system 100 may be used more than once, in other embodiments, testing modules 202A-D may be configured to be reused or replaced. For example, testing modules 202A-D may be configured with seals 306, such as rubber barrier having one or more slits that open when impinged by sample transport 220, and then reseal once sample transport 220 has moved away. Testing modules 202A-D may be configured to be replaceable individually or in a replacement set. For example, testing modules 202A-D may configured to be replaced individually, or replaced as a group.

As described further below, in an embodiment, BV testing system 100 may also include a transceiver 242 for wirelessly communication with third party systems, a network adapter, a power source 250, such as a battery pack, and the like. In some embodiments, BV testing system may include a global positioning system (GPS) to allow third parties, such as hospitals, clinics, and the like, to monitor the location of the BV testing system 100. Such monitoring may allow medical personnel and others to track cases of bacterial vaginosis, or other medical conditions detected by BV testing system 100.

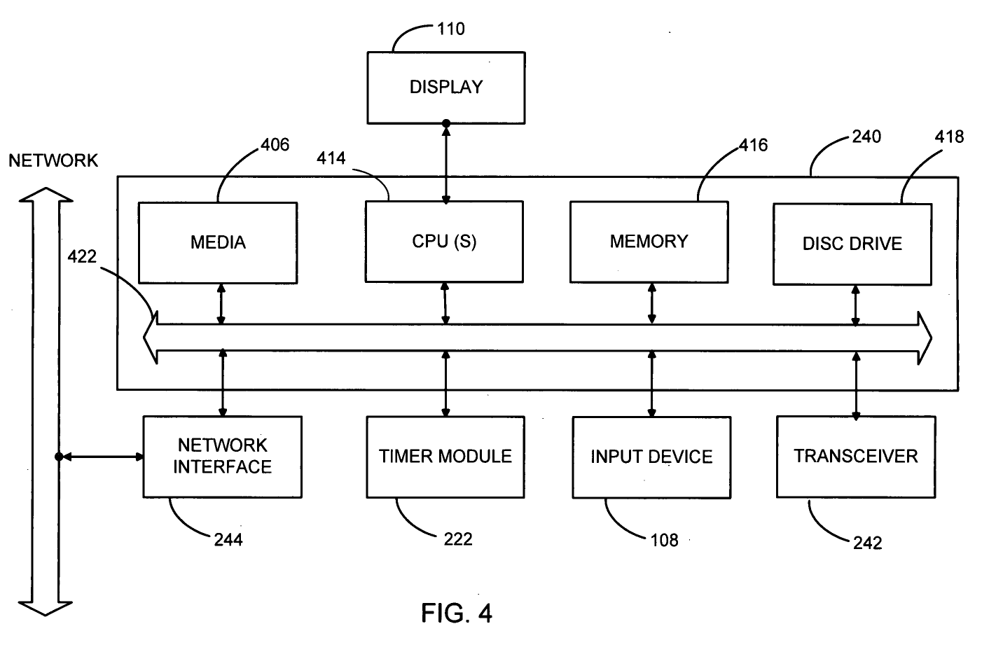

As shown in FIG. 4, BV testing system 100 may include testing driver 204. Testing driver 204 may include one or more processors 414, GPUs, or the like, and one or more volatile memory storage areas such as memory 416. Memory 416 may be any type of memory such as Random Access Memory (RAM), non-volatile memory, volatile memory, and the like. Memory 416 may be configured to store one or more computer programs for execution by processor 414, such as a program configured to test for BV in accordance with embodiments of the present invention, examples of which are described herein. Testing driver 204 may further include one or more nonvolatile memory storage areas such as disk drives 418 and removable media 406, and an electrical bus 422 interconnecting the above components coupled to external systems via a wireless connection and/or though data port 118.

In one set of embodiments, disk drive 418 and/or removable media 406 may be configured to store a representation of one or more bacterial vaginosis testing sequences, and the like in accordance with embodiments of the present invention. Removable media 406 may correspond to any type of nonvolatile storage media or device, such as optical media (e.g., CD-ROM, DVD-ROM, Blu-Ray Disc, HD-DVD, etc.), nonvolatile flash media (e.g., CompactFlash, SD, MemoryStick. etc.), removable hard disks, or the like.

Memory 416, disk drives 418, and removable media 406 are examples of tangible computer readable media for storage of data, audio/video files, computer programs, and the like. Other types of tangible media include floppy disks, USB drives, removable hard disks, optical storage media such as CD-ROMS and bar codes, semiconductor memories such as flash memories, read-only-memories (ROMS), battery-backed volatile memories, networked storage devices, and the like.

In various embodiments, testing driver 204 may be communicatively coupled with one or more wired user input devices 108 and/or one or more wireless user input devices via transceiver 242. Wired user input device 108 may be any type of input device capable of communication via a wired connection/protocol (e.g., USB, Firewire, PS/2. etc.) such as a keyboard, mouse, a trackball, a track pad, a joystick, a game controller, a drawing tablet, microphone, and the like. In various embodiments, user input device 108 allows a user to select objects, icons, text and the like that graphically appear on a display 110 via one or more input commands such as a button click or the like. In some embodiments, user input device 108 may include controls (e.g., buttons, switches, etc.) physically located on testing body 112.

Transceiver 242 may be any type of input device capable of communication via a wireless connection/protocol (e.g., infrared, radio frequency (RF), Bluetooth, etc.) such as a wireless remote control, a wireless keyboard, wireless mouse, a wireless game controller, and the like.

In further embodiments, one or more network/data interfaces 244 may be provided for communicatively coupling testing driver 204 with other devices. For example, network/data interface 244 may couple testing driver 204 with one or more computers on a computer network, a FireWire device, a Satellite cable connection, an optical cable, a wired-cable connection, or the like. Network/data interface 244 may include an Ethernet-based network adapter, modem (e.g., telephone, satellite, cable, ISDN, etc.), (asynchronous) digital subscriber line (DSL) unit, FireWire interface, USB interface, CableCard™ interface, and the like. Further, network/data interface 244 may be physically integrated on a motherboard of testing driver 204, or may be a software program such as soft DSL or the like.

Network/data interface 244 may be coupled with one or more data storage locations such as web servers and the like via one or more communication networks, including local-area networks; wide-area networks, such as the Internet or cellular data networks; and virtual private networks. In some embodiments, network/data interface 244 may be configured to receive analog or digital image data to be decoded and output to viewers via display 110. Display 110 may be a passive window type display and/or any output device configured to display an image such as a monitor, television, display on a portable device such as a cellular phone, and the like.

Embodiments of BV testing system 100 may implement processors 414 and peripheral components, such as memory 416, network/data interface 244, graphics, audio and video peripherals as separate components, components combining two or more of these functions, and/or software programs implementing some or all of the functionality of these peripherals.

In an embodiment, testing driver 204 may also include software that enables communications over communication network/data interface 412 such as the SMB/CIFS, NFS, ZeroConf, HTTP, TCP/IP, RTP/RTSP protocols, wireless application protocol (WAP), and the like. In alternative embodiments of the present invention, other communications software and transfer protocols may also be used, for example IPX, UDP or the like.

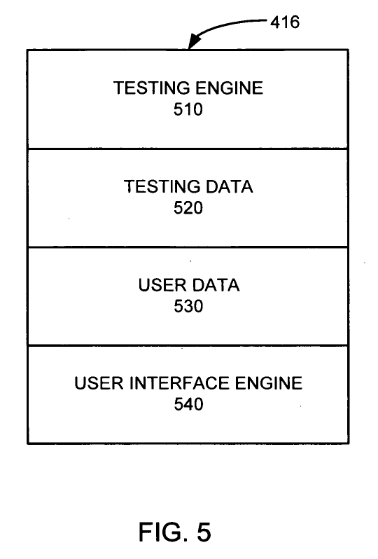

FIG. 5 is a high-level illustration of memory 416. Memory 416 may include a BV testing engine 510, testing data 520, user data 530, and a user interface engine 540. As described further below, BV testing engine 510 is a software engine that facilitates a user in testing for bacterial vaginosis.

In one embodiment, BV testing engine 510 may be a stand alone program included in an operating system, library, daemon, or background process application, may be a plug-in program used to modify and enhance the operation of other programs, or may incorporated as part of other programs source code. For example, BV testing engine 510 may be configured to be incorporated into, operate in conjunction with, or modify the operation of visual display and user interface programs such as Microsoft Windows, OS X user interface programs, Linux user interface, and the like.

In an embodiment, testing data 520 may be digital data including but not limited to virtually any data related to medical testing of a sample from a patient. For example testing data may include testing data such as fluid pH, Gram stain results, and the like from a bacterial vaginosis test sequence described herein. Testing data 520 may also include data pertaining to the location of the test, the temperature of the sample, and other data associated with a test. For example, testing data 520 may include the test results, instructions for administering the test, feedback forms, GPS information, date of the test, how long until the BV testing system 100 needs servicing, power data, test results, physician instructions to the patient, trend analysis of multiple tests, and the like.

User data 530 may be any suitable data capable of identifying a user. User data 530 may contain information pertaining to the user's age, sex, and the like. In some embodiments, user data may include other types of data. For example, user data may include the medical records of a patient using BV testing system 100, bio-metric data, etc.

In one embodiment, memory 416 includes a user-interface engine 540. User-interface engine 540 may be any suitable interface used for example to operate BV testing engine 510, BV testing system 100, and the like. User interface 540 may be a graphical user interface (GUI) such as a browser program that provides icons and other graphical information to a user, for example on display 110, to operate a software program such as BV testing engine 510. User interface engine 540 may also include other interfaces such as a command line interface. In further embodiments, user interface 540 may be omitted. Instead, BV testing engine 210 may be configured to interact directly with an operating system or a software application. In this embodiment, BV testing engine 210 may operate transparently to the user and rely on the user interface of the operating system or the application.

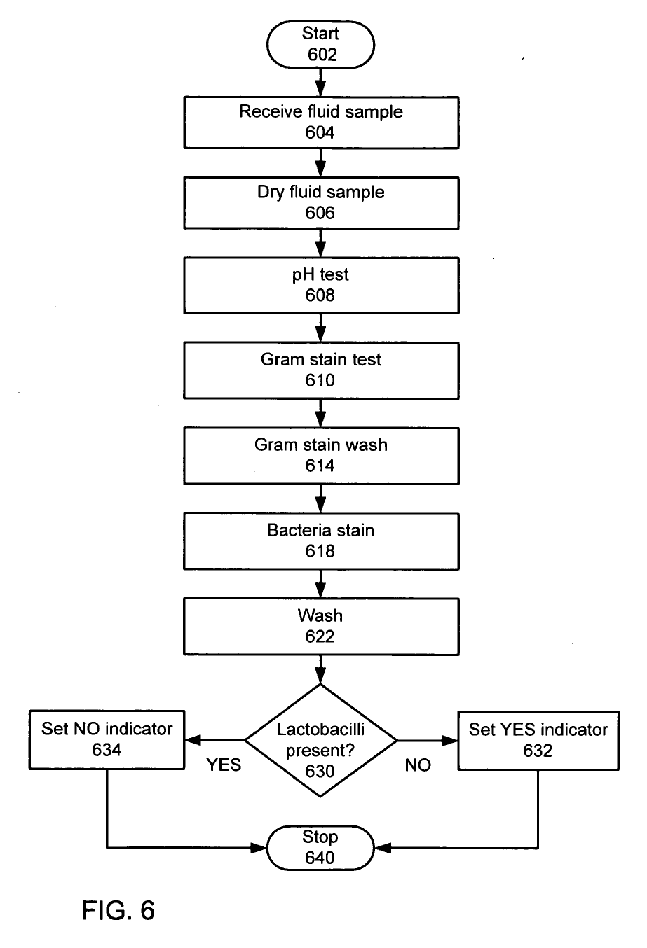

FIG. 6 is a high-level flow diagram illustrating an embodiment of a method 600 of testing for bacterial vaginosis. Method 600 may be entered into at step 602 when a user, for example, initiates the bacterial vaginosis test.

At step 604, method 600 obtains user data to determine the user. For example, method 600 obtains user information from user data 530. In another embodiment, bio-metric data is obtained from a user to authenticate the user.

At step 606, the fluid sample is dried. For example, method 600 places the fluid sample in testing module 202A for about two minutes to dry. For example, sample transport may move fluid sample to module 202B to test the pH. Module 202B may be lined with a pH moist strip that can be graced by the swab. At step 608, the pH moist strip may be configured to indicate an acidic pH with a purple color or basic pH with a red color. Alternately, module 202B may include a methyl red solution may include a blue coloring. The sample may remain in this area for one minute. If the solution turns a first indicator color such as purple, the methyl red has indicated a pH of 4.5 to 5.0 (more acidic). Otherwise the solution will turn another color such as green, indicating a higher pH. In this embodiment, a patient may use view ports 116 to see if the solution has turned color.

If the sample has turned to the first indicator color, at step 610, method 600 performs a gram strain test. For example, sample transport 220 moves sample into testing module 220C containing a CV I ionic solution or commonly “gram's iodine”. In one embodiment, the solution will remain here for about two minutes or longer to stain any gram positive bacteria a second indicator color. At step 614, the sample is washed in a basic solution such as ethanol, and the like, to determine if the sample is gram positive or gram negative, or neither. For example, sample transport 220 may move sample into a second compartment of testing module 202C to immerse the sample in an ethanol wash. If the second indicator color remains, then the sample is gram positive, however, if the indicator color does not change (e.g., stays green), the sample is gram negative, or the patient may have another condition such as a yeast infection.

At step 618, the fluid sample is moved via sample transport 120 to testing module 220D to stain any gram-negative bacteria. For example, fluid sample may be exposed to a safarnin to stain the gram negative bacteria. After about two minutes, at step 622 the sample is moved via sample transport 120 to a separate compartment of testing module 202D for a wash to remove the stain. In one embodiment, the fluid sample is washed in water. If at step 630, if there are no gram negative bacteria in the sample, the wash removes the stain and the sample becomes clear, and at step 632 method 600 sets a NO indicator (e.g., flag, display, etc.) indicating that the patient likely does not have bacterial vaginosis. However, if gram negative bacteria are present, a predetermined indicator remains from the safarnin stain, such as a safarnin stain indicator color, method 600 sets a YES indicator (e.g., flag, display, etc.) at step 634 indicating the patient may have bacterial vaginosis due to the presence of gram-negative bacteria.

To further verify a diagnosis, the patient may observe if the pH is acidic via a view port 116. If the sample is both acidic (indicator color indicates acidity) and gram positive (sample retains gram-positive indicator), the patient most likely does not have bacterial vaginosis. However, if the sample indicates gram negative, and pH is less acidic, then the patient likely has bacterial vaginosis. If the swab does no indicate the presence of gram-positive or gram-negative bacteria, yeast mostly likely inhabits the vaginal cavity, and therefore, the patient may have a yeast infection. In one embodiment, such pH and stain indications may be processed by test driver 204 and results displayed on display 110 either having the sample viewable through a view port 116 and/or display 110, or via an image displayed on display 110 (e.g. a “+” sign). Method 600 ends at step 640.

Further aspects of embodiments of the invention are illustrated in the attached figures. Additional embodiments can be envisioned to one of ordinary skill in the art after reading the attached documents. In other embodiments, combinations or sub-combinations of the above disclosed invention can be advantageously made. The block diagrams of the architecture and flow charts are grouped for ease of understanding. However it should be understood that combinations of blocks, additions of new blocks, re-arrangement of blocks, and the like are contemplated in alternative embodiments of the present invention.

The specification and drawings are, accordingly, to be regarded in an illustrative rather than a restrictive sense. It will, however, be evident that various modifications and changes may be made thereunto without departing from the broader spirit and scope of the invention.

Any of the above described steps may be embodied as computer code on a computer readable medium. The computer readable medium may reside on one or more computational apparatuses and may use any suitable data storage technology.

The present invention can be implemented in the form of control logic in software or hardware or a combination of both. The control logic may be stored in an information storage medium as a plurality of instructions adapted to direct an information processing device to perform a set of steps disclosed in embodiment of the present invention. Based on the disclosure and teachings provided herein, a person of ordinary skill in the art will appreciate other ways and/or methods to implement the present invention.

A recitation of “a”, “an” or “the” is intended to mean “one or more” unless specifically indicated to the contrary.

All patents, patent applications, publications, and descriptions mentioned above are herein incorporated by reference in their entirety for all purposes. None is admitted to be prior art.

What is claimed is:

1. A portable system for bacterial vaginosis testing, the system comprising:
a portable enclosure;
a plurality of testing modules disposed within the portable enclosure and disposed about a testing path, wherein each testing module is configured to perform at least one portion of a bacterial vaginosis test on a vaginal fluid sample from a patient;
a sample transport member disposed within the enclosure and configured to support the vaginal fluid sample and position the vaginal fluid sample within each of the plurality of testing modules; and
a timer module coupled to the sample transport member, the timing module configured to move the sample transport over a predetermined time period about the testing path.

2. The system of claim 1, wherein the enclosure comprises a sample port extending therein, wherein the sample port is configured to allow the fluid sample to passed through the enclosure and delivered to the sample transport.

3. The system of claim 1, wherein the enclosure comprises one or more viewing ports extending through the enclosure such that the vaginal fluid sample is visible along at least a portion of the testing path to a user.

4. The system of claim 1, wherein the testing path is a circular path or non-circular path.

5. The system of claim 1, wherein the timer module is a linear timer or non-linear timer.

6. The system of claim 5, wherein the timer module is a mechanical spring driven timer.

7. A method for a user self-test for bacterial vaginosis, the method comprising;
providing a fluid sample to a portable testing device, wherein the portable testing device comprises at least two testing modules disposed along a testing path and a test transport member configured to support the fluid sample;
moving the transport member containing a fluid sample between the at least two testing modules along the testing path.
processing the fluid sample with the at least two testing modules;
determining from at least some of the bacteria in the processed fluid sample whether the patient has bacterial vaginosis; and
providing a display to a user thereof indicating the results of the fluid sample processing.

8. The method of claim 7, wherein moving the transport member comprises rotating the transport member about an axis of a timer module configured to move the fluid sample along the testing path.

9. The method of claim 7, wherein processing the fluid sample comprises determining at a first of the two testing modules the acidity of the fluid sample.

10. The method of claim 7, wherein processing the fluid sample comprises determining at least at a second of the at least two testing modules whether the fluid sample is gram positive or gram negative.

11. The method of claim 7, wherein providing a display comprises code for providing a visual indicator of whether the patient has or does not have bacterial vaginosis.

12. The method of claim 7, wherein processing the fluid sample comprises determining from the fluid sample whether vaginal flora of the patient contains Lactobacilli.

13. The method of claim 7, moving the transport member at a predetermined rate along the testing path such that the fluid sample is allowed to process for a predetermined time between the at least two testing modules.

14. A computer program product having a computer readable medium storing a set of code modules which when executed by a processor of a computer system cause the processor to process fluid samples from a patient to determine if the patient has bacterial vaginosis, the computer readable medium comprising:
code for providing a fluid sample to a portable testing device, wherein the portable testing device comprises at least two testing modules disposed along a testing path and a test transport member configured to support the fluid sample;
code for moving the transport member and the fluid sample between the at least two testing modules along the testing path.
code for processing the fluid sample with the at least two testing modules;
code for determining from bacteria within the processed fluid sample whether the patient has bacterial vaginosis; and
code for providing a display to a user thereof indicating the results of the fluid sample processing.

15. The computer program product of claim 14, wherein code for moving the transport member comprises code for rotating the transport member about an axis of a timer module configured to move the transport member along the testing path.

16. The computer program product of claim 14, wherein the code processing the fluid sample comprises code for determining the acidity of the fluid sample.

17. The computer program product of claim 14, wherein code for processing the fluid sample comprises code for determining whether the sample is gram positive or gram negative.

18. The computer program product of claim 14, code for providing a display comprises code for providing a visual indicator of whether the patient has bacterial vaginosis.

19. The computer program product of claim 14 wherein code for processing the fluid sample comprises code for determining whether vaginal flora of the patient contains Lactobacilli.

20. The computer program product of claim 14, comprising code for moving the fluid sample at a predetermined rate along the testing path such that the fluid sample is allowed to process for a predetermined time between the at least two testing modules.
